(19) United States
(12) Patent Application Publication
(10) Pub. No.: US 2003/0118659 A1
(43) Pub. Date: Jun. 26, 2003

(54) Antigen-specific induction of peripheral immune tolerance

(76) Inventors: Thomas J. August, Baltimore, MD (US); Kam W. Leong, Baltimore, MD (US); Robert Georgantas, Lockport, IL (US)

Correspondence Address: BANNER & WITCOFF, 1001 G STREET N W, SUITE 1100, WASHINGTON, DC, 20001, US

(21) Appl. No.: 10/162,652

(22) Filed: Jun. 6, 2002

Related U.S. Application Data
(63) Continuation of application No. 09/958,100, now abandoned.
Application No. 09/958,100 is a 371 of international application No. PCT/US00/09461, filed on Apr. 10, 2000.
(60) Provisional application No. 60/128,420, filed on Apr. 8, 1999.

(51) Int. Cl. A61K 48/00; A61K 9/16; A61K 9/50

(52) U.S. Cl. 424/491; 514/44

(57) ABSTRACT

Fas ligand (CD95L) induces apoptosis in activated T cells through the process of Activation Induced Cell Death (AICD). Gelatin nanoparticles are virus sized gelatin-protein-DNA complexes which can encapsulate multiple DNA vectors and proteins, and which are thought to act by increasing in vivo transfection of antigen presenting cells. By injecting mice with gelatin nanoparticles containing a murine Fas ligand (CD95L) DNA vector and a β-galactosidase (LacZ) model antigen vector, the T cell response specific for β-gal was ablated without effecting the response to a secondary antigen. In effect, this “tolerization” injection induced antigen specific peripheral tolerance in study mice, and is applicable to the treatment of autoimmune diseases when self-antigens such as Myelin Basic Protein are co-delivered with the fas ligand.

### TECHNICAL FIELD OF THE INVENTION

This invention is related to the area of immune diseases. More particularly it relates to autoimmune diseases, allergic diseases, and transplantation rejection.

### BACKGROUND OF THE INVENTION

Immunologic tolerance to self antigens is a necessary mechanism for protecting an organism from destruction by its own immune system. When this mechanism malfunctions, allowing self-reactive immune cells to proliferate, an autoimmune disease develops within the host. A number of diseases such as Multiple Sclerosis, Lupis, Myathenia Gravis, and Rheumatoid Arthritis have been shown to result from loss of self-tolerance in T and B lymphocytes.

Fas ligand (CD95L) plays a substantial role in both the regulation [1] [2] and effector function [3] of the immune system, as well as in the maintenance of immunologic privilege [4] [5] [6-8]. T lymphocytes utilize CD95L to induce apoptosis in target cells, and also become susceptible to CD95L after antigen stimulation through the processes of Activation Induced Cell Death (AICD). [9] [10] T lymphocytes reactive with self-proteins play a substantial role in autoimmune pathology [11]. For example, Myelin reactive T cells have been demonstrated in patients with Multiple Sclerosis [12] [13] [14] and in the experimental autoimmune enchephelomyclitis mouse and rat models have been shown to be both pathogenic and able to transfer disease between syngeneic animals. [15-18]

There is a continuing need in the art for additional methods and tools for treating autoimmune diseases, as well as allergic diseases and transplantation rejection.

### SUMMARY OF THE INVENTION

It is an object of the present invention to provide a method of ablating or reducing a pathological immune response, such as an autoimmune response, an allergic response, or transplantation rejection.

It is another object of the present invention to provide compositions of nanospheres, antigen presenting cells, nucleic acid expression vectors, or liposomes useful for ablating or reducing an immune response.

These and other objects of the invention are achieved by providing a method in which nucleic acids are administered to a mammal. The nucleic acids encode a first and a second protein which are a molecule which induces apoptosis or inactivation of T cells and a pathogenic antigen, respectively. Antigen presenting cells express both the first protein and the second protein Antigen-specific T cells are activated by the antigen presenting cells which express the pathogenic antigen. Activated antigen-specific T cells are killed or inactivated by the first protein expressed by the antigen presenting cells. Thus an immune response to the antigen can be ablated.

Also provided are nanospheres which comprise at least one nucleic acid molecule encoding a first and a second protein. The first protein induces apoptosis or inactivation of T cells, and the second protein comprises a pathogenic antigen. The pathogenic antigen is capable of activating antigen-specific T cells. The first protein is capable of killing or inactivating activated antigen-specific T cells.

Also provided by the present invention is a composition comprising a liposome which comprises at least one nucleic acid molecule encoding a first and a second protein. The first protein induces apoptosis or inactivation of T cells, and the second protein comprises a pathogenic antigen. The pathogenic antigen is capable of activating antigen-specific T cells and the first protein is capable of killing or inactivating activated antigen-specific T cells.

According to another embodiment of the invention a composition is provided which comprises a nucleic acid vector encoding a first and a second protein. The first protein induces apoptosis or inactivation of T cells, and the second protein comprises a pathogenic antigen. The pathogenic antigen is capable of activating antigen-specific T cells and the first protein is capable of killing or inactivating activated antigen-specific T cells.

In yet another embodiment of the invention a composition comprising a genetically engineered cell is provided. The cell comprises at least one nucleic acid molecule encoding a first and a second protein. The first protein induces apoptosis or inactivation of T cells, and the second protein comprises a pathogenic antigen. The pathogenic antigen is capable of activating antigen-specific T cells and the first protein is capable of killing or inactivating activated antigen-specific T cells.

The present invention thus provides the art with methods and compositions useful for treating autoimmune diseases, transplantation rejection, and allergic diseases.

### BRIEF DESCRIPTION OF THE DRAWINGS

FIG. 1. Fas ligand/Lac Z co-delivery abrogates the CTL immune response to β-gal. Mice were injected and re-injected at 2 weeks with saline alone (●), 1 μg of p43-FasL DNA in nanoparticles (▪), 1 μg of p43-cLacZ in nanoparticles (♦), 1 μg of LacZ/FasL in nanoparticles (▴), or 1 μg of p43-cLacZ in nanoparticles in one leg and 1 μg of p43-FasL in nanoparticles in the opposite leg (▾). T cells were isolated from the spleen and lymph nodes of the mice and assayed for β-gal specific cytotoxicity.

FIG. 2. Systemic abrogation of the β-gal CTL immune response by Fas ligand/LacZ co-delivery. Mice were three times at two week intervals with saline alone (●), 50 μg of p43-FasL naked DNA in the left leg (▪), 50 μg of p43-FasL naked DNA in the left leg and LacZ/FasL DNA nanoparticles (2 μg of DNA total) in the right leg (♦), 100 μg of p43-FasL naked DNA in the left leg (▴), or 100 μg of p43-FasL naked DNA in the left leg and LacZ/FasL DNA nanoparticles (2 μg of DNA total) in the right leg (▾). T cells were isolated from the spleen and lymph nodes of the mice and assayed for β-gal specific cytotoxicity.

FIG. 3. Antigen specific induction of tolerance by LacZ/FasL nanoparticles. Mice were injected at 0, 2, and 4 weeks with saline (●, p43-cLacZ nanoparticles (1 μg) (▪), LacZ/FasL nanoparticles (2 μg) (♦), pcHA vector (50 μg) in the left leg (▴), or p43-cLacZ nanoparticles (1 μg) in the right leg and pcHA vector (50 μg) in the left leg (▾). T cells harvested from the spleen and lymph nodes of and were assayed for CTL response against β-gal (FIG. 3A) or to HA (FIG. 3B).

FIG. 4. Requirement for co-delivery of Fas ligand and LacZ vectors. Mice were injected at 0, 2, and 4 weeks with saline (▾), 50 μg of naked p43-LacZ (●), 50 μg of naked p43-LacZ in the left leg and LacZ/FasL DNA nanoparticles (2 μg of DNA total) in the right leg (▪), 50 μg of naked p43-cLacZ (♦), a mixture of 50 μg of p43-cLacZ and 50 μg of p43-FasL (▴). Two weeks after the final injections, T cells were harvested from the spleen and lymph nodes and were assayed for CTL response against β-gal.

FIG. 5. The effects of LaZ/FasL nanoparticle injection on β-Gal immunity of pre- and post-immunized mice. Mice were injected with saline (●), three times with 1 μg of p43-LacZ nanoparticles (▪), three times with 50 μg of naked p43-LacZ (♦), twice with 2 μg of LacZ/FasL nanoparticles followed by three timer with 1 μg of p43-LacZ in nanoparticles (▴), or three times with 1 μg of p43-LacZ nano-particles followed by twice with 2 μg of LacZ/fasL nanoparticles (▾). AL1 immunization injections were given at 2-week intervals and tolerizing injections were given at 1-week intervals. Three weeks after the final injection, T cells were isolated from the spleen and lymph nodes and were assayed for β-Gal-specific cytotoxicity.

### DETAILED DESCRIPTION

It is a discovery of the present inventors that co-expression within individual cells of a pathogenic antigen and a cell protein which kills or inactivates activated T cells leads to activation and antigen-induced cell death (AICD) in reactive T cells. It is believed that interaction of T cells with protein epitopes bound in the MHC class I and II molecules of antigen presenting cells (APCs) leads to antigen-specific activation of T cells. These activated T cells then interact with a cell protein which inactivates or kills activated T cells, inducing these T cells to undergo apoptosis. It is a further finding of the inventors that nanospheres formed by coacervation of more than one coding sequence are efficient means of achieving co-tranfection, without requiring special digenic constructs.

Pathogenic antigens according to the present invention are those against which a pathological immune reaction is directed. Thus the antigen can be, for example, an allergen, an autoantigen, or a transplantation antigen. Autoimmune antigens are relevant to several diseases, including Multiple Sclerosis, Rheumatoid Arthritis, Myasthenia gravis, and Lupis Erithmatosis. Other diseases in which an autoimmune component has been implicated are Insulin Dependent Diabetes Mellitus, and Crohn's disease.

Nucleic acids encoding the pathogenic antigen of choice may include a targeting signal directing the protein to the MHC class II antigen presentation system. One particularly preferred targeting system is the LAMP-1 targeting system. See U.S. Pat. No. 5,633,234, which is expressly incorporated herein. The LAMP targeting system guides proteins to the endosomal/lysosomal system for processing and presentation. LAMP-mediated lysosomal targeting of antigens has been shown to increase the immune reaction to the antigen through a mechanism of increased antigen presentation to CD4+ T helper cells. In contrast, no particular targeting system is required to achieve antigen presentation in the MHC class I system. Presentation via the MHC class I system activates CD8+ cytotoxic T cells. Activation and subsquent deletion of the CD4+ and CD8+ populations abrogates both antibody and cellular immune responses.

Nucleic acids can be used which encode any cellular protein which induces apoptosis or inactivation of T cells. The cellular protein can be a surface protein or a secreted protein for which the activated T cells express a receptor. Preferably expression of the receptor is up-regulated upon activation of the T cells. Suitable cellular proteins include, without limitation, Fas ligand and TNFα. Secreted proteins such as cytokines and chemokines can be used as well.

Ablation of an immune response includes reduction, alleviation, amelioration and total elimination. The immune response can be evaluated by measuring any relevant biological component of the immune system or by evaluating symptoms of a patient or mammal being treated. See, Chapter 2, “The Induction, Measurement, and Manipulation of the Immune response,” in Immunobiology, the immune system in health and disease, Janeway, Travers, Hunt, and Walport, Current Biology Limited, NY, 1997. Preferably a reduction of at least 25% is achieved. More preferably a reduction of at least 50%, 75%, 80%, 85%, 90%, or 95% is achieved.

Administration of nucleic acids according to the invention can be according to any procedure known in the art. While delivery of nanospheres which coacervate both coding sequences is desired, other delivery systems as are known in the art can be used, including but not limited to liposomes, viral particles, naked DNA, gene guns, and the like. Cells which have been transfected ex vivo can also be used for delivery of the nucleic acids to a mammal. Cells can be antigen presenting cells or muscle cells. Preferably they are autologous. Although applicants do not wish to be bound by any particular theory or mechanism of operation, it appears that co-tranfection of cells with both components, the coding sequence for the pathogenic antigen and the coding sequence for the cellular protein which induces apoptosis or inactivation, is required. Thus methods which maximize the probability of cotransfection are preferred. The two coding sequences can be on a single molecule of nucleic acid or they can be physically associated in a complex, for example. Those of skill in the art will be able to choose the precise configuration which is most convenient for the particular application at hand.

Uptake of the nucleic acids by antigen presenting cells can be accomplished as is known in the art. See Akbari et al., J. Exp. Med. 189:169-177; Casares et al, J. Exp. Med. 186:1481-1486; Condon et al, Nature Medicine 2:1122-1128; and Porgador et al, J. Exp. Med. 188:1075-1082. If a targeting moiety is desired to increase the number of antigen presenting cells which take up the DNA, one can target N418, CD83, MHC class II, CD80 and CD86 (B7.1 and B7.2) and CD40. For example, antibodies or antibody portions which are specific for these cell makers can be attached to the nanospheres.

Expression vectors which are useful according to the present invention are molecules which will ensure transcription and translation of the encoded proteins. Appropriate expression vectors are known in the art for mammalian cells and in particular for human cells. The promoters used may be constitutive or regulated. The expression units for the apoptosis-inducing protein and the pathogenic antigen may be on one or two distinct molecules.

Liposomes are well known in the art. Any technique may be used for making the liposomes at the choice of the skilled artisan. The liposomes can co-encapsulate two distinct expression vectors or one vector which encodes both the pathogenic antigen and the apoptosis-inducing protein.

Modes of administration to the mammal or patient can be by any conventional route. These include, but are not limited to subcutaneous, intramuscular, intravenous, intraperitoneal, oral, intranasal, intrabronchial, and scarification. While it is believed that the present method works by in vivo uptake of the DNA directly by antigen presenting cells, it is possible that other cells such as muscle cells take up the DNA as well.

Nanopsheres typically are coacervates of a positively charged and a negatively charged polymer. Nanospheres which employ nucleic acids as the negatively charged polymer can employ, for example, gelatin, chitosan, or derivatives thereof as the positively charged polymer. The nanospheres preferably are less than 5 μm and more preferably less than 3 μm. A population of nanospheres can be sized. Whereas there may be some variability in the size of the population the size of at least 50%, 75%, 85%, or 90% of the population can be applied as characterizing the population.

The above disclosure generally describes the present invention. A more complete understanding can be obtained by reference to the following specific examples which are provided herein for purposes of illustration only, and are not intended to limit the scope of the invention.

### EXAMPLE 1

Injection of Mice with LacZ/FasL Nanoparticles

Immunization of mice with gelatin nanoparticles containing a β-gal encoding p43-cLacZ vector had been previously shown to produce a large antigen specific immune reaction in mice. Also, protein molecules such as cytokines, which effect the immune system, could be encapsulated and co-delivered with the DNA vector nanoparticles resulting in a shift in the immune response toward a Th1 or Th2 bias. [25]

Since our theoretical model for antigen specific deletion of T cells required that individual cells be co-transfected with both antigen-encoding and fas ligand-encoding vectors, we tested the effect on the immune system of injecting mice with LacZ/FasL nanoparticles as compared to LacZ only nanoparticles.

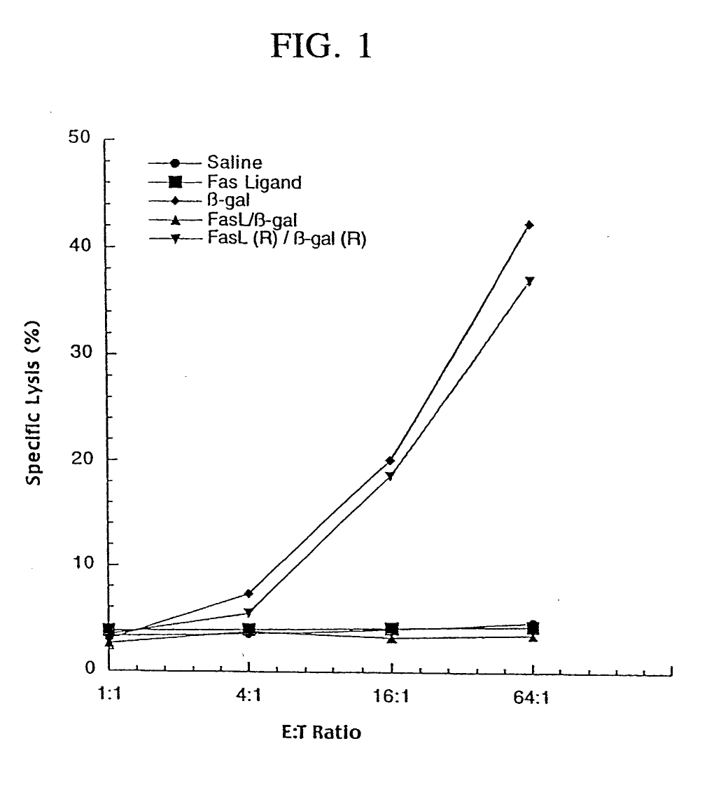

We found that a normal immune response to the β-gal protein is generated when mice are immunized with LacZ vector alone or with LacZ in one leg and Fas ligand in the opposite leg (FIG. 1). When co-encapsulated LacZ and Fas ligand are injected, the immune reaction against β-gal is reduced to background reactivity, similar to the group injected with saline alone. Injection of Fas ligand nanoparticles alone had no effect on the immune response. Thus, the co-delivery of a Fas ligand expression vector and an antigen-encoding vector leads to abrogation of the CTL response to the antigen.

Methods: DNA Vectors

The p43-cLacZ vector encoding the E. coli β-galactosidase gene has been previously described [26]. The Fas ligand mRNA sequence was obtained by PCR of the gene from the pCI-FasL vector, which was the generous gift of Dr. Berry Burns. Primers for PCR of the gene sequence, sense 5′-3′ and antisense 5′-3′, contained EcoR I linkers. The p43-FasL vector encoding the C57B/6 mouse mRNA sequence was constructed by ligation of the DNA fragment into the EcoR I restriction site of the p43 DNA vector, and the resulting construct was sequenced by the Biochemistry Core facility to assure sequence integrity. Both vectors were propagated in DH5 cells, and were purified using the Qiagen EndoFree Plasmid Kits. Purified plasmid DNA was assayed for bacterial LPS contamination.

Materials: Mice

6-8 week old BALB/c mice were purchased from the Charles Rivers Laboratories. All protocols for animal use were approved.

Methods: Immunization of Animals

Gelatin nanoparticles were prepared as described [24, 25] and DNA loading level was quantitated by Flourometry after digestion with 2.5% trypsin to release the DNA from the gelatin. Mice were immunized by intramuscular injection in tibialis anterior muscle of the rear legs with naked DNA or gelatin nanoparticles suspended in sterile saline solution.

Methods: Cytotoxic T Cell Assay

Lymph nodes and spleens were harvested from mice and T cells isolated by non-adherence to nylon wool as previously described and were assayed for antigen specific cytotoxicity by chromium release assay as previously described [27] [25]. Chromium release was quantitated on a Packard Top Count NXT, and results expressed as Counts×min-1 (CPM)

### EXAMPLE 2

Systemic Abrogation of T cell Activity

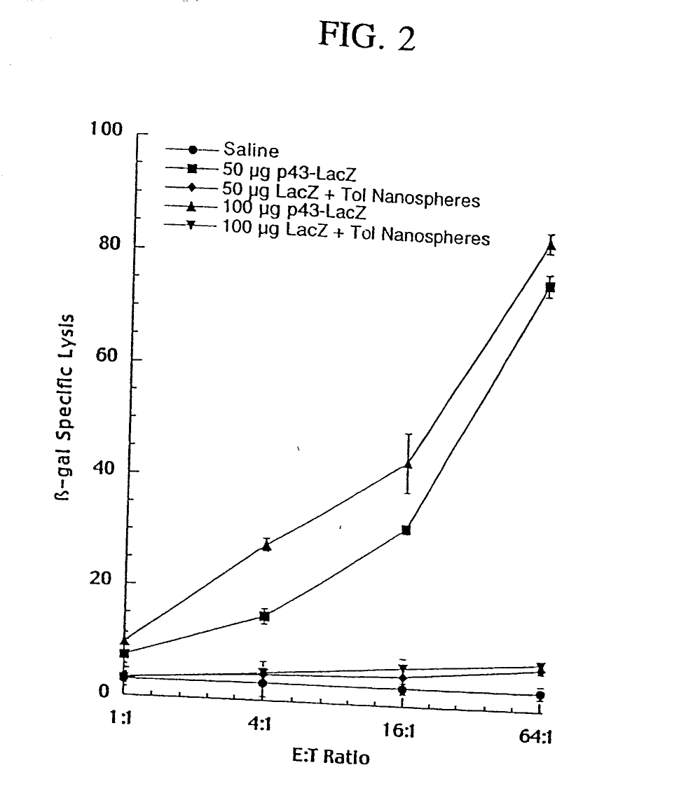

Although activated APCs become refractory to apoptosis and specifically to Fas ligand mediated apoptosis by down-regulation the Fas receptor, the possibility was raised that the expression of the Fas ligand on the surface of transfected APC's was inducing auto-apoptosis of the APC, thereby killing or inactivating the APC before it was able to interact with T cells, thereby preventing it from ever inducing an immune reaction to the antigen. To rule out this possibility, mice were immunized in one leg with a high dose of naked p43-cLacZ and in the opposite leg with LacZ/FasL nanoparticles (FIG. 2). Our reasoning being that if Fas ligand expression was simply causing auto-apoptosis of transfected cells, the immune response generated in the opposite leg injected with naked p43c-cLacZ would not effected. On the other hand, expression of antigen and the Fas ligand by individual APCs and muscle cells, which would display both activating antigen and AICD-inducing fas ligand to T cells, should lead to systemic deletion of β-gal reactive T cells.

Mice injected with naked p43-cLacZ alone developed strong CTL responses to β-gal, whereas those injected with naked p43-cLacZ in one leg and LacZ/FasL nanoparticles in the opposite leg developed responses only slightly higher than that of the group injected with saline alone. Thus, the co-delivery of fas ligand- and LacZ-encoding vectors leads to systemic abrogation of the immune response by a mechanism other than inducing apoptosis in the transfected APCs.

### EXAMPLE 3

Abrogation of T Cell Activity in Mice Immunized with Antigen Before or After Tolerization.

LacZ/FasL nanoparticles systemically abrogate the β-gal specific immune response when injected at the same time as an immunization, clearly effecting the development of an immune response. However, the effectiveness against pre-existing T cells or against antigen-specific precursor T cells was not known. To answer these questions, mice were immunized three times with p43-cLacZ nanoparticles before or after injection of LacZ/FasL nanoparticles.

### EXAMPLE 4

Antigen Specific Induction of T Cell Tolerance

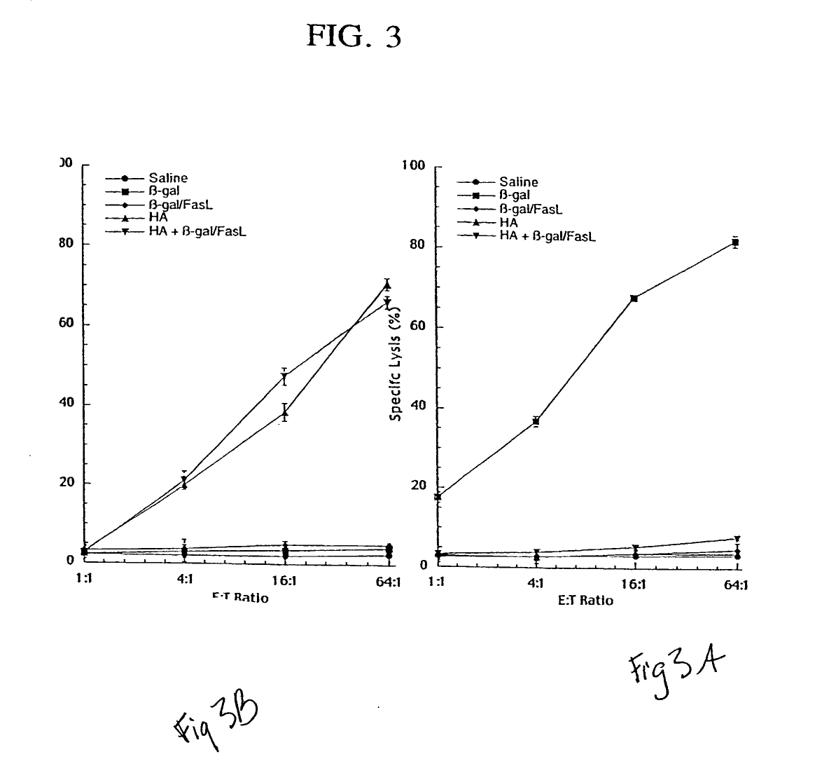

Having demonstrated that LacZ/FasL tolerization nanoparticles (“Tol nanospheres”) abrogate both the local and systemic immune reaction to β-gal, we next asked if the tolerization injection would affect the immune response to a second antigen injected at a separate site. Mice were injected in one leg with the pcHA vector and in the opposite leg with LacZ/FasL nanoparticles. While co-delivery of LacZ and Fas ligand resulted in abrogation of the β-gal specific response (FIG. 3A), the immune response to influenza hemmaglutinin (HA) was not affected (FIG. 3B). These data demonstrate that the deletion of T cells induced by the LacZ/FasL nanoparticles is antigen-specific, and that the phenomenon does not affect secondary antigens that are not co-expressed with the fas ligand.

### EXAMPLE 5

Requirement of Vector Co-Delivery

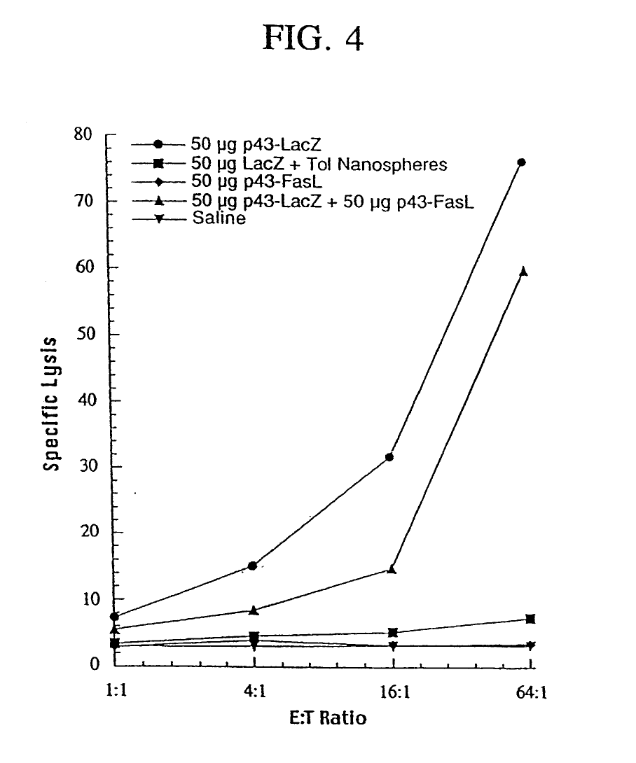

Our model of the tolerization system assumed that a co-delivery system is necessary to ensure co-expression of antigen and Fas ligand within individual cells. To test if co-delivery was in fact required (FIG. 4), mice were injected with naked p43-cLacZ (50 μg), naked p43-cLacZ (50 μg) and LacZ/FasL nanoparticles (2 μg), naked p43-FasL (50 μg), or a mixture of naked p43-cLacZ (50 μg) and p43-FasL (50 μg). Harvested T cells were assayed for CTL activity against β-gal. The saline and p43-FasL immunization groups did not show β-gal-specific lysis activity. The p43-cLacZ group developed a strong response to β-gal. The LacZ/FasL (Tol nanospheres) with p43-cLacZ nanoparticles resulted in the abrogation of the response. The group injected with naked LacZ and FasL vectors showed only a small decrease in the immune response compared to the decrease observed in the nanoparticle group. Therefore, although a small decrease in the immune response was observed after injection of the naked vectors, co-delivery is a requirement for the abrogation of the immune response to an antigen.

In this study we have attempted to induce peripheral tolerance of T lymphocytes by taking advantage of the phenomena of Activation Induced Cell Death, in which activated T cells become susceptible to induction of apoptosis by the fas ligand. An immune response to an antigen is initiated when an APC expressing antigen in surface bound MHC class molecules interacts with a CD4+ T helper lymphocytes. The CD4+ cell becomes activated and at the same time displays the CD40 ligand (CD154), which fully activates the APC. The APC is then able to activate CD8+ T cytotoxic lymphocytes through display of antigen on surface-bound MHC class I molecules. We speculated that co-expressing antigen and fas ligand on individual cells might cause antigen-specific activation of T cells and induction of AICD through interaction with CD95L.

We co-encapsulated two separate DNA vectors, one encoding the CD95L and one the β-galactosidase model antigen, within gelatin nanoparticles. Gelatin nanoparticles are a coacervate of DNA and gelatin that have previously been demonstrated to have utility in vaccine delivery and in gene therapy applications. See U.S. Ser. Nos. 08/657,913, filed Jun. 7, 1996, 60/071,679, filed Jan. 16, 1998; and 60/071,746, filed Jan. 16, 1998, which are expressly incorporated herein. Our theory of CD95L induced tolerance required that both the antigen and CD95L genes be co-expressed in individual cells. We tested nanoparticles for the ability to to deliver two distinct nucleic acid constructs to the same cell without the need of building elaborate digeneic DNA constructs.

We have shown that T cells reactive to a specific antigen are deleted in mice injected with nanoparticles encoding both the specific antigen and a surface molecule which induces apoptosis.

### EXAMPLE 6

Multiple Sclerosis Antigens: Myelin Basic Protein

An excellent MS model exists in the SJL/J strain of mice which mirrors the human disease in both its pathology and immunological characteristics. This disease model, called experimental allergic encephalomyelitis (EAE), is used to characterize the tolerization technique for prevention and treatment of multiple sclerosis.

The myelin basic protein mRNA sequence from the SJL/J mouse was cloned by RT-PCR into a mammalian expression vector. It was subsequently subcloned into a tPA-TmC chimera mammalian expression vector. Expression of myelin basic protein with the fas ligand is used to delete mouse T cells reactive with myelin basic protein, as described above for β-galactosidase.

### EXAMPLE 7

Inhibition of CTL Activity of Pre- or Postimmunized Mice

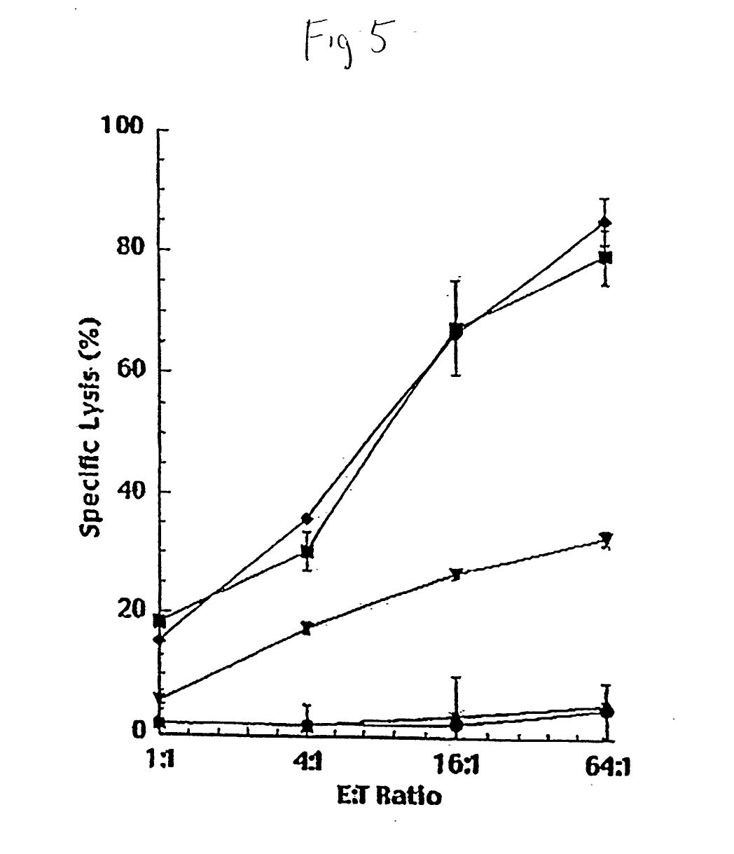

LacZ/FasL DNA nanoparticles systemically inhibited the β-Gal-specific CTL response when injected into mice at the same time as administration of the immunizing DNA. We have also tested the effectiveness of the tolerization method in mice with T cells already present from a previous antigen challenge or against rechallenge with antigen after the tolerization injection (FIG. 5). Mice were injected twice with LacZ/FasL. DNA nanoparticles (2 μg of DNA) either 2 weeks before or 2 weeks after they were immunized three times with 1 μg of p43-cLacZ nanoparticles. There was no significant CTL response in mice injected twice with LacZ/FasL nanoparticles before immunization with nanoparticles containing p43-cLacZ DNA alone, yielding T cell reactivity similar to that of mice injected with saline alone. Mice immunized three times with p43-cLacZ nanoparticles and then injected with LacZ/FasL nanoparticles 7 and 14 days after the last immunization showed a great decrease in, but not complete abrogation of, T cell reactivity compared with mice injected with 1 μg of p43-cLacZ nanoparticles or with 50 μg of naked p43-cLacZ. Within the time frame of this experiment, injection of the LacZ/FasL nanoparticles prevented the development of a β-Gal-specific T cell response to antigen challenge after the mice had been tolerized. Also, LacZ/fasL nanoparticles were able to markedly reduce, but not completely abrogate, the T cell reactivity to antigen generated by immunization before tolerization injections were administered.

### REFERENCES

1. Van Parijs, L. A., A. K., Homeostasis and self-tolerance in the immune system: turning lymphocytes off. Science, 1998. 280(5361): p. 243-248.

2. Wang, J. L., M. J., Molecules involved in cell death and peripheral tolerance. Current Opinion in Immunology, 1997. 9(6): p. 818-825.

3. Podack, E. R., Functional significance of two cytotoxic pathways of cytotxic T lymphocytes. Journal of Leukocyte Biology, 1995. 57: p. 548-552.

4. Greil, R. E., A. Villunger, A., On The Role And Significance Of Fas (Apo-1/CD95) Ligand (FasL) Expression In Immune Privileged Tissues And Cancer Cells Using Multiple Myeloma As A Model. Leukemia & Lymphoma, 1998. 31(5-6): p. 477.

5. Green, D. R. W., Carl F., Fas-ligand: Privilege and peril. Proceedings of the National Academy of Sciences, 1997. 94: p. 5989-5990.

6. Griffith, T. S. B., T. Fletcher, S. M. Green, D. R. Ferguson, T. A, Fas ligand-induced apoptosis as a mechanism of immune privilege. Science, 1995. 270(5239): p. 1189-1192.

7. Griffith, T. S. F., Thomas A., The role of FasL-induced apoptosis in immune privilege. Immunology Today, 1997. 18(5): p. 240-244.

8. Griffith, T. S. Y., X. Herndon, J. M. Green, D. R. Ferguson, T. A., CD95-induced apoptosis of lymphocytes in an immune privileged site induces immunological tolerance. Immunity, 1996. 5(1): p. 7-16.

9. Green, D. R. S., David W., Activation-induced apoptosis in lymphocytes. Current opinion in Immunology, 1994. 6: p.476-487.

10. Wong, B. A., Joseph Choi, Yongwon, T cell receptor signals enhance susceptibility to Fas-mediated apoptosis. journal of experimental medicine, 1997. 186(11): p. 1939.

11. Bach, J. F. K., S. Vanendert, P. M., Are there unique autoantigens triggering autoimmune diseases. Immunological Reviews, 1998. 164: p. 139-155.

12. Vrethem, M.D., C. Ekerfelt, C. Forsberg, P. Danielsson, O. Ernerudh, J., CD4 and CD8 lymphocyte subsets in cerebrospinal fluid and peripheral blood from patients with multiple sclerosis, meningitis and normal controls. Acta Neurologica Scandinavica, 1998. 97(4): p. 215-220.

13. de Rosbo, N. K. H., Michael Ben-Nun, Avrabam, Predominance of the autoimmune response to myelin oligodendrocyte glycoprotein (MOG) in multiple sclerosis: reactivity to the extracellula domain of MOG is directed against three main regions. European journal of immunology, 1997. 27(11): p. 3059.

14. Boiko, A. N. F., O. O., Multiple sclerosis—molecular and cell mechanisms. Molecular Biology, 1995. 29(4): p. 413-426.

15. Patterson, P. Y., Transfer of allergic encephalitis in rates by means of lymph node cells. Journal of experimental medicine, 1960. 111: p. 119-133.

16. Trotter, J., et al., Characterization fT cell lines and clones from SJL/J and (BALB/cxSJL/J)F1 mice specific for myelin basic protein. Journal of Immuology, 1985.134: p. 2322-2327.

17. Ben-Nun, A., H. Wererle, and I. R. Cohen, The rapid isolation of clonable antigen-specific lymphocyte lines cabable of mediateing autoimmune encephalomyelitis. European Journal of Immunology, 1981. 11: p. 195-199.

18. Lyons, J. -A., M. -L. Zhao, and R. B. Fritz, Pathogenesis of acute passive murine encephalomyelitis I. Importance of host-derived cells as determines by kinetic analysis. Journal of neuroimmunology, 1998. 86: p. 92-103.

19. Maecker, H. U., D T. DeKruyff, R H. Levy, S., Cytotoxic T cell responses to DNA vaccination: Dependence on antigen presentation via class II MHC. Journal of Immunology, 1998. 161(12): p. 6532-6536.

20. Akbari, O. P., N. Garcia, S. Tascon, R. Lowrie, D. Stockinger, B., DNA vaccination: Transfection and activation of dendritic cells as key events for immunity. Journal of Experimental Medicine, 1999. 189(1): p. 169-177.

21. Chattergoon, M. A. R., Tara M. Weiner, David B., Specific Immune Induction Following DNA-Based Immunization Through In Vivo Transfection and Activation of Macrophages/Antigen-Presenting Cells. Journal of immunology, 1998. 160(12): p. 5707.

22. Porgador, A. I., K R Iwasaki, A Barber, B H Restifo, N P Germain, R N., Predominant Role For Directly Transfected Dendritic Cells In Antigen Presentation To CD8(+)T Cells After Gene Gun Immunization. Journal of Experimental Medicine, 1998. 188(6): p. 1075-1082.

23. Donnelly, J. J., et al., DNA vaccines. Annual Review of Immunology, 1997. 15: p. 617-48.

24. Truong-Le, V. L. W., Scott M. Schweibert, Erik Mao, Hai-Quan Guggino, William B. August, J. Thomas Leong, Kam W., Gene transfer by DNA-gelatin nanospheres. Archives of Biochemistry and Biophysics, 1999. 361(1): p. 47-56.

25. Truong-Le, V. L., et al., DNA-Gelatin nanoarticles as vaccines: immunologic effects of co-delivered cytokine proteins and protection against ebola virus. in preparation,.

26. Troung, V. L., in Pharmacology and Molecular Science. 1998, Maryland: Baltimore.

27. Coligan, J. E., in Current Protocols in Immunology. 1994.

All articles, texts, and patents cited in this application are expressly incorporated herein by reference.

1. A method of reducing an antigen-specific immune response, comprising:
administering nucleic acids to a mammal, wherein the nucleic acids encode a first and a second protein, wherein the first protein induces apoptosis or inactivation of T cells, and the second protein is a pathogenic antigen, whereby an immune response to the antigen in the mammal is reduced.

2. The method of claim 1 wherein antigen presenting cells in the mammal take up the nucleic acids, whereby the antigen presenting cells express the first protein and the second protein, whereby antigen-specific T cells are activated and killed.

3. The method of claim 1 wherein the nucleic acids are administered to the mammal by delivery of antigen presenting cells which comprise the nucleic acids.

4. The method of claim 1 wherein the nucleic acids are administered to the mammal by delivery of muscle cells which comprise the nucleic acids.

5. The method of claim 1 wherein the first and the second proteins are encoded by separate nucleic acid molecules.

6. The method of claim 1 wherein the pathogenic antigen is an allergen.

7. The method of claim 1 wherein the pathogenic antigen is an autoimmune antigen.

8. The method of claim 7 wherein the autoimmune antigen is one to which the mammal mounts an immune response.

9. The method of claim 1 wherein the pathogenic antigen is a transplantation antigen.

10. The method of claim 2 wherein the first protein is expressed on the surface of the antigen presenting cells.

11. The method of claim 2 wherein the first protein is secreted by the antigen presenting cells.

12. The method of claim 1 wherein the first protein is Fas ligand.

13. The method of claim 1 wherein the first protein is Tnf-α.

14. The method of claim 1 wherein the second protein is lysosomally targeted.

15. The method of claim 1 wherein the second protein is not lysosomally targeted.

16. The method of claim 2 wherein receptors for the first protein on T cells are up-regulated upon activation of the T cells.

17. The method of claim 1 wherein the nucleic acid molecules are coacervated in nanospheres.

18. The method of claim 5 wherein the nucleic acid molecules are coacervated in nanospheres.

19. The method of claim 14 wherein a CD4+ T helper cell population is killed.

20. The method of claim 15 wherein a CD8+ cytolytic T cell poopulation is killed.

21. The method of claim 1 wherein the antigen is myelin basic protein.

22. A composition comprising a nanosphere which comprises at least one nucleic acid molecule encoding a first and a second protein, wherein the first protein induces apoptosis or inactivation of activated T cells, and the second protein comprises a pathogenic antigen, wherein the pathogenic antigen is capable of activating antigen-specific T cells.

23. A composition comprising a liposome which comprises at least one nucleic acid molecule encoding a first and a second protein, wherein the first protein induces apoptosis or inactivation of activated T cells, and the second protein comprises a pathogenic antigen, wherein the pathogenic antigen is capable of activating antigen-specific T cells.

24. A composition comprising a nucleic acid expression vector encoding a first and a second protein, wherein the first protein induces apoptosis or inactivation of activated T cells, and the second protein comprises a pathogenic antigen, wherein the pathogenic antigen is capable of activating antigen-specific T cells.

25. A composition comprising an antigen presenting cell which comprises at least one nucleic acid molecule encoding a first and a second protein, wherein the first protein induces apoptosis or inactivation of activated T cells, and the second protein comprises a pathogenic antigen, wherein the pathogenic antigen is capable of activating antigen-specific T cells, wherein the antigen presenting cell expresses both the first and the second proteins.

26. The composition of claim 22, 23, or 25 wherein the first and the second proteins are encoded by separate nucleic acid molecules.

27. The composition of claim 22, 23, 24, or 25 wherein the pathogenic antigen is an allergen.

28. The composition of claim 22, 23, 24, or 25 wherein the pathogenic antigen is an autoimmune antigen.

29. The composition of claim 22, 23, 24, or 25 wherein the pathogenic antigen is a transplantation antigen.

30. The composition of claim 22, 23, 24, or 25 wherein the first protein is a cell surface protein.

31. The composition of claim 22, 23, 24, or 25 wherein the first protein is Fas ligand.

32. The composition of claim 22, 23, 24, or 25 wherein the first protein is TNF-α.

33. The composition of claim 22, 23, 24, or 25 wherein the second protein is lysosomally targeted.

34. The composition of claim 22, 23, 24, or 25 wherein the second protein is not lysosomally targeted.

35. The composition of claim 22, 23, 24, or 25 wherein receptors for the first protein on T cells are up-regulated upon activation of the T cells.

36. The composition of claim 33 wherein a CD4+ T helper cell population is targeted.

37. The composition of claim 34 wherein a CD8+ cytolytic-T cell poopulation is targeted.

38. The composition of claim 22, 23, 24, or 25 wherein the antigen is myelin basic protein.
